(19) United States
(12) Patent Application Publication
(10) Pub. No.: US 2025/0177990 A1
(43) Pub. Date: Jun. 5, 2025

(54) METHODS AND SYSTEMS FOR ALIGNING A SAMPLE PLATE

(71) Applicant: Foundation Medicine, Inc., Boston, MA (US)

(72) Inventor: Steven LEIKEFELD, Penzberg (DE)

(73) Assignee: Foundation Medicine, Inc., Boston, MA (US)

(21) Appl. No.: 18/844,564

(22) PCT Filed: Sep. 19, 2022
(86) PCT No.: PCT/US2022/076669

§ 371 Date: Sep. 6, 2024

Related U.S. Application Data
(60) Provisional application No. 63/318,526, filed on Mar. 10, 2022.

(51) Int. Cl. B01L 9/00; C12N 15/10

(52) U.S. Cl. CPC B01L 9/52; C12N 15/1006; B01L 2200/025

(57) ABSTRACT

The present disclosure relates generally to an alignment mechanism, and more specifically to techniques for requiring correct loading of a sample plate into a scientific instrument or system (e.g., a DNA extractor system). An alignment device comprises: a base portion (102) configured to be disposed along an edge (310) of a target location (300); and a protruded portion (104) connected to the base portion (102), wherein, when the base portion (102) is affixed along the edge (310) of the target location (300), the protruded portion (104) is configured to: cover at least partially a comer (301) of the target location (300), and align with a lateral surface (202a) of a sample plate (200) to require the sample plate (200) to be loaded into the target location (300) in a correct orientation.

### CROSS-REFERENCE TO RELATED APPLICATIONS

This application claims the benefit of U.S. Provisional Application No. 63/318,526, filed Mar. 10, 2022, which is hereby incorporated by reference in its entirety.

### FIELD OF THE INVENTION

The present disclosure relates generally to an alignment mechanism, and more specifically to techniques for ensuring correct loading of a sample plate into a scientific instrument or system (e.g., a DNA extractor system).

### BACKGROUND

In various scientific processes, a sample plate (e.g., containing one or more biological samples from one or more subjects) may need to be loaded in a scientific instrument/system for further processing and analysis. For example, a DNA extractor system can comprise one or more receptacles for accommodating one or more sample plates. After the sample plates are loaded, the DNA extractor system may further process and analyze the biological samples in the sample plates.

Because a sample plate is often symmetrical in shape, there is a risk that the sample plate may be loaded into a scientific instrument/system in the wrong orientation. For example, if the sample plate is rectangular, it may be rotated by 180 degrees and may still fit into the receptacle of the scientific instrument. This may cause the samples to be analyzed incorrectly, e.g., using incorrect reagents by disposing the reagents in an incorrectly-oriented sample plate, etc. The loading error may be difficult to discover and expensive to remedy. For example, if a sample plate is loaded in the wrong orientation by a technician into a DNA extractor system, the error may only be discovered after sequencing a few days later due to a ˜50% gender discordance. Once the error is discovered, all patient reports would immediately go on hold. If the gender discordance cannot be solved, either a new sample must be requested by the physician, or a new extraction has to be made.

### BRIEF SUMMARY OF THE INVENTION

Disclosed herein are exemplary devices, apparatuses, systems, and methods for ensuring correct loading of a sample plate (e.g., in the correct orientation). With the alignment mechanism, it is not possible to load the sample plate in an incorrect orientation. The alignment device permits the sample plate to be loaded only if it is in the correct orientation and may ensure that the sample plate is not being loaded when it is in an incorrect orientation. As a result, the alignment mechanism may eliminate the occurrence of loading errors, which are difficult to discover and expensive to remedy, and may improve the accuracy of the scientific instrument or system. The alignment mechanism also may eliminate the need for a second laboratory technician to check and confirm the correct loading of the sample plates into the scientific instrument, thus reducing the cost associated with operating the scientific instrument. Further, the attachment of the alignment device does not alter the functioning of the scientific instrument or system.

An exemplary alignment device comprises: a base portion (102) configured to be disposed along an edge (310) of a target location (300) and a protruded portion (104) connected to the base portion (102), wherein, when the base portion (102) is disposed along the edge (310) of the target location (300), the protruded portion (104) is configured to cover at least partially a corner (301) of the target location (300), and align with a lateral surface (202a) of a sample plate (200) to require the sample plate (200) to be loaded at the target location (300) in a correct orientation.

In some embodiments, the base portion (102) is attached along the edge (310) of the target location (300).

In some embodiments, the protruded portion (104) is of a triangular shape.

In some embodiments, an angle (105) of the triangular shape is 45°.

In some embodiments, the base portion (102) is configured to be disposed along the edge (310) of the target location (300) via one or more fasteners (109a, 109b).

In some embodiments, the one or more fasteners (109a, 109b) comprise one or more bolt/nut sets, screws, rivets, welds, or any combination thereof.

In some embodiments, the base portion (102) comprises one or more apertures (108a, 108b) configured to receive the one or more fasteners (109a, 109b).

In some embodiments, the one or more apertures (108a, 108b) are configured to allow for a location of the alignment device to be adjustable.

In some embodiments, the base portion (102) is configured to be disposed along the edge (310) of the target location via adhesive.

In some embodiments, the target location (300) includes a recessed receptacle disposed in a DNA extractor system.

In some embodiments, the sample plate (200) is adapted to contain one or more biological samples.

In some embodiments, the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

In some embodiments, the alignment device comprises a rigid material that is resistant to corrosion and/or rust.

In some embodiments, the alignment device comprises stainless steel.

In some embodiments, the alignment device comprises plastic or resin.

In some embodiments, the alignment device is formed via one or more additive manufacturing techniques.

In some embodiments, the base portion (102) and the protruded portion (104) are parts of a single component.

An exemplary receptacle of a scientific instrument for accommodating a sample plate comprises: a bottom (304) configured to fit a bottom (204) of the sample plate (200); a protruded portion (104) covering at least partially a corner (301) of an opening of the receptacle, wherein when the sample plate (200) is loaded in a correct orientation, the protruded portion (104) is configured to align with a lateral cutoff (202a) of the sample plate (200) to allow the sample plate to be placed on the bottom (304) of the receptacle; and wherein when the sample plate (200) is loaded in an incorrect orientation, the protruded portion is configured to require the sample plate (200) to be loaded on the bottom (304) in a correct orientation.

In some embodiments, the protruded portion (104) is part of an alignment device comprising a base portion (102) configured to be disposed along an edge (310) of the receptacle.

In some embodiments, the base portion (102) is configured to be attached along the edge (310) of the receptacle.

In some embodiments, the base portion (102) is configured to be disposed along the edge (310) of the receptacle via one or more fasteners (109a, 109b).

In some embodiments, the one or more fasteners (109a, 109b) comprise one or more bolt/nut sets, screws, rivets, welds, or any combination thereof.

In some embodiments, the base portion (102) comprises one or more apertures (108a, 108b) configured to receive one or more fasteners (109a, 109b).

In some embodiments, the one or more apertures (108a, 108b) are configured to allow for a location of the alignment device to be adjustable.

In some embodiments, the base portion (102) is configured to be disposed along the edge (310) of the receptacle via adhesive.

In some embodiments, the alignment device comprises a rigid material that is resistant to corrosion.

In some embodiments, the alignment device comprises stainless steel.

In some embodiments, the alignment device comprises plastic or resin.

In some embodiments, the alignment device is formed via one or more additive manufacturing techniques.

In some embodiments, the base portion (102) and the protruded portion (104) are parts of a single component.

In some embodiments, the protruded portion (104) is an integral part of the receptacle.

In some embodiments, the bottom (304) of the receptacle is rectangular.

In some embodiments, the protruded portion (104) is of a triangular shape.

In some embodiments, an angle (105) of the triangular shape is 45°.

In some embodiments, the receptacle is configured to be disposed in a DNA extractor system.

In some embodiments, the sample plate (200) is adapted to contain one or more biological samples.

In some embodiments, the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

An exemplary method for installing an alignment device (100) for enabling a sample plate (200) to be loaded at a target location (300) in a correct orientation, wherein the alignment device (100) comprises a base portion (102) and a protruded portion (104) extending from the base portion (102), comprises: disposing the alignment device (100) adjacent to the target location (300) such that the protruded portion (104) of the alignment device (100) covers at least a portion (301) of the target location (300).

In some embodiments, the protruded portion (104) is of a triangular shape.

In some embodiments, an angle (105) of the triangular shape is 45°.

In some embodiments, disposing the alignment device (100) comprises disposing the base portion (102) along an edge (310) of the target location (300).

In some embodiments, the base portion (102) is disposed along the edge (310) via adhesive.

In some embodiments, the base portion (102) is disposed along the edge (310) via one or more fasteners (109a, 109b).

In some embodiments, the one or more fasteners (109a, 109b) comprise one or more bolt/nut sets, screws, rivets, welds, or any combination thereof.

In some embodiments, the base portion comprises one or more apertures (108a, 108b) configured to receive the one or more fasteners (109a, 109b).

In some embodiments, the one or more apertures (108a, 108b) are configured to allow for a location of the alignment device (100) to be adjustable.

In some embodiments, disposing the alignment device (100) comprises: inserting one fastener from the one or more fasteners (109a, 109b) into each of the one or more apertures (108a, 108b); moving the alignment device (100) relative to the one or more fasteners (109a, 109b) in each of the one or more apertures (108a, 108b); and securing, in response to the alignment device (100) being at a desired position relative to the target location (300), the position of the alignment device (100).

In some embodiments, the base portion (102) is configured to attach along the edge (310) of the target location (300).

In some embodiments, the target location (300) includes a recessed receptacle disposed in a DNA extractor system.

In some embodiments, the sample plate (200) is adapted to contain one or more biological samples.

In some embodiments, the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

In some embodiments, the alignment device (100) comprises a rigid material that is resistant to corrosion and/or rust.

In some embodiments, the alignment device (100) comprises stainless steel.

In some embodiments, the alignment device (100) comprises plastic or resin.

In some embodiments, the alignment device (100) is formed via one or more additive manufacturing techniques.

In some embodiments, the base portion (102) and the protruded portion (104) are parts of a single component.

An exemplary DNA extractor comprises: a target location (300); and an alignment device (100) comprising: a base portion (102) configured to be disposed along an edge (310) of the target location (300); a protruded portion (104) connected to the base portion (102), wherein, when the base portion (102) is disposed along the edge (310) of the target location (300), the protruded portion (104) is configured to: cover at least partially a corner (301) of the target location (300), and align with a lateral surface (202a) of a sample plate (200) to to require the sample plate (200) to be loaded at the target location (300) in a correct orientation.

In some embodiments, the protruded portion (104) is of a triangular shape.

In some embodiments, an angle (105) of the triangular shape is 45°.

In some embodiments, the base portion (102) is configured to be disposed along the edge (310) of the target location (300) via one or more fasteners (109a, 109b).

In some embodiments, the one or more fasteners (109a, 109b) comprise one or more bolt/nut sets, screws, rivets, welds, or any combination thereof.

In some embodiments, the base portion (102) comprises one or more apertures (108a, 108b) configured to receive the one or more fasteners (109a, 109b).

In some embodiments, the one or more apertures (108a, 108b) are configured to allow for a location of the alignment device (100) to be adjustable.

In some embodiments, the base portion (102) is configured to be disposed along the edge (310) of the target location (300) via adhesive.

In some embodiments, the base portion (102) is configured to attach along the edge (310) of the target location (300).

In some embodiments, the target location (300) includes a recessed receptacle disposed in a DNA extractor system.

In some embodiments, the target location (300) includes a container configured to receive the sample plate (200).

In some embodiments, the target location (300) includes a planar surface configured to receive the sample plate (200).

In some embodiments, the sample plate (200) is adapted to contain one or more biological samples.

In some embodiments, the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

In some embodiments, the alignment device (100) comprises a rigid material that is resistant to corrosion and/or rust.

In some embodiments, the alignment device (100) comprises stainless steel.

In some embodiments, the alignment device (100) comprises plastic or resin.

In some embodiments, the alignment device (100) is formed via one or more additive manufacturing techniques.

In some embodiments, the base portion (102) and the protruded portion (104) are parts of a single component.

An exemplary method for installing an alignment device (100) for requiring correct loading of a sample plate (200) into a target location (300), wherein the target location (300) comprises one or more apertures (306a-d) along an edge (310) of the target location (300) and wherein the alignment device (100) comprises: a base portion (102) and a protruded portion (104) connected to the base portion (102), may comprise: removing one or more fasteners of a first length from the one or more apertures; and disposing the base portion (102) along the edge (310) of the target location (300) via the one or more apertures (306a-d) and one or more fasteners of a second length (109a, 109b) longer than the first length such that the protruded portion (104) covers at least partially a corner (301) of the target location (300) and aligns with a lateral surface (202a) of the sample plate (200) to require the sample plate (200) to be loaded into the target location (300) in a correct orientation.

In some embodiments, the one or more fasteners of the first length are screws of the first length.

In some embodiments, the one or more fasteners of the second length are screws of the second length.

In some embodiments, the protruded portion (104) is of a triangular shape.

In some embodiments, an angle (105) of the triangular shape is 45°.

In some embodiments, the base portion (102) comprises one or more apertures (108a, 108b) configured to receive the one or more fasteners of the second length (109a, 109b).

In some embodiments, the one or more apertures (108a, 108b) are configured to allow for a location of the alignment device (100) to be adjustable.

In some embodiments, the target location (300) includes a recessed receptacle disposed in a DNA extractor system.

In some embodiments, the target location (300) includes a container configured to receive the sample plate (200).

In some embodiments, the target location (300) includes a planar surface configured to receive the sample plate (200).

In some embodiments, the sample plate (200) is adapted to contain one or more biological samples.

In some embodiments, the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

In some embodiments, the alignment device (100) comprises a rigid material that is resistant to corrosion and/or rust.

In some embodiments, the alignment device (100) comprises stainless steel.

In some embodiments, the alignment device (100) comprises plastic or resin.

In some embodiments, the alignment device (100) is formed via one or more additive manufacturing techniques.

In some embodiments, the base portion (102) and the protruded portion (104) are parts of a single component.

An exemplary method for extracting deoxyribonucleic acid (DNA) molecules from a sample from a subject using a DNA extraction device comprises: providing the sample in a sample plate (200); disposing an alignment device (100) comprising a base portion (102) and a protruded portion (104) extending from the base portion (102) adjacent to a target location (300) on the DNA extraction device such that the protruded portion (104) of the alignment device (100) covers at least a portion (301) of the target location (300); placing the sample plate (200) at the target location (300) such that the protruded portion (104) is aligned with a lateral cutoff (202a) of the sample plate (200); extracting a plurality of nucleic acid molecules, including at least one DNA molecule, from the sample in the sample plate (200); ligating one or more adapters onto one or more nucleic acid molecules from the plurality of nucleic acid molecules; amplifying the one or more ligated nucleic acid molecules from the plurality of nucleic acid molecules; capturing amplified nucleic acid molecules from the amplified nucleic acid molecules; and sequencing, by a sequencer, the captured nucleic acid molecules to obtain a plurality of sequence reads that represent the captured nucleic acid molecules, wherein one or more of the plurality of sequencing reads overlap one or more gene loci within a subgenomic interval in the sample.

In some embodiments, the protruded portion (104) is of a triangular shape.

In some embodiments, an angle (105) of the triangular shape is 45°.

In some embodiments, disposing the alignment device (100) comprises disposing the base portion (102) along an edge (310) of the target location (300).

In some embodiments, the base portion (102) is disposed along the edge (310) via adhesive.

In some embodiments, the base portion (102) is disposed along the edge (310) via one or more fasteners (109a, 109b).

In some embodiments, the one or more fasteners (109a, 109b) comprise one or more bolt/nut sets, screws, rivets, welds, or any combination thereof.

In some embodiments, the base portion (102) comprises one or more apertures (108a, 108b) configured to receive the one or more fasteners.

In some embodiments, the one or more apertures (108a, 108b) are configured to allow for a location of the alignment device (100) to be adjustable.

In some embodiments, disposing the alignment device (100) comprises: inserting one fastener from the one or more fasteners (109a, 109b) into each of the one or more apertures (108a, 108b); moving the alignment device (100) relative to the one or more fasteners (109a, 109b) in each of the one or more apertures (108a, 108b); and securing, in response to the alignment device (100) being at a desired position relative to the target location (300), the position of the alignment device (100).

In some embodiments, disposing the alignment device (100) comprises attaching the base portion (102) along the edge (310) of the target location.

In some embodiments, the target location (300) includes a recessed receptacle disposed in a DNA extractor system.

In some embodiments, the sample plate (200) is adapted to contain one or more biological samples.

In some embodiments, the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

In some embodiments, the alignment device (100) comprises a rigid material that is resistant to corrosion and/or rust.

In some embodiments, the alignment device (100) comprises stainless steel.

In some embodiments, the alignment device (100) comprises plastic or resin.

In some embodiments, the alignment device (100) is formed via one or more additive manufacturing techniques.

In some embodiments, the base portion (102) and the protruded portion (104) are parts of a single component.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

Various aspects of the disclosed methods, devices, and systems are set forth with particularity in the appended claims. A better understanding of the features and advantages of the disclosed methods, devices, and systems will be obtained by reference to the following detailed description of illustrative embodiments and the accompanying drawings, of which:

FIG. 1A provides a non-limiting example of an exemplary alignment device adapted to aid in loading of a sample plate, in accordance with some embodiments.

FIG. 1B provides a non-limiting example of an exemplary alignment device adapted to aid in loading of a sample plate, in accordance with some embodiments.

FIG. 1C provides a non-limiting example of a top view of an exemplary alignment device adapted to aid in loading of a sample plate, in accordance with some embodiments.

FIG. 1D provides a non-limiting example of a bottom view of an exemplary alignment device adapted to aid in loading of a sample plate, in accordance with some embodiments.

FIG. 1E provides a non-limiting example of a side view of an exemplary alignment device adapted to aid in loading of a sample plate, in accordance with some embodiments.

FIG. 1F provides a non-limiting example of a top view of an exemplary alignment device adapted to aid in loading of a sample plate, in accordance with some embodiments.

FIG. 2 provides a non-limiting example of an exemplary sample plate, in accordance with some embodiments.

FIG. 3A provides a non-limiting example of an exemplary receptacle of a scientific instrument or system that is configured to accommodate a sample plate, in accordance with some embodiments.

FIG. 3B provides a non-limiting example of the exemplary alignment device installed on the scientific instrument or system that is adapted to aid in loading of sample plates, in accordance with some embodiments.

FIG. 3C provides a non-limiting example of the exemplary alignment device installed on the scientific instrument or system that is adapted to aid in loading of sample plates, in accordance with some embodiments.

FIG. 4A provides a non-limiting example of how the alignment device permits the sample plate to fit into the receptacle when the sample plate is in the correct orientation, in accordance with some embodiments.

FIG. 4B provides a non-limiting example of how the alignment device permits the sample plate to fit into the receptacle when the sample plate is in the correct orientation, in accordance with some embodiments.

FIG. 5A provides a non-limiting example of how the alignment device ensures the sample plate fits into the receptacle only when the sample plate is in a correct orientation, in accordance with some embodiments.

FIG. 5B provides a non-limiting example of how the alignment device ensures the sample plate fits into the receptacle only when the sample plate is in a correct orientation, in accordance with some embodiments.

FIG. 6 illustrates an exemplary process for installing the alignment device for ensuring correct loading of a sample plate into a recessed receptacle, in accordance with some embodiments.

FIG. 7 illustrates an exemplary process for installing the alignment device for ensuring correct loading of a sample plate into a target location, in accordance with some embodiments.

FIG. 8 illustrates an exemplary process for sequencing a sample of nucleic acid molecules using a correctly loaded sample plate, in accordance with some embodiments.

### DETAILED DESCRIPTION

Disclosed herein are exemplary devices, apparatuses, systems, and methods for requiring correct loading of a sample plate (e.g., in the correct orientation). An exemplary alignment device can comprise a base portion configured (e.g., structured) to be disposed (e.g., affixed or attached) along an edge of a target location, such as a recessed receptacle, a container configured to receive the sample plate, or a surface area on which the sample plate can be placed. The alignment device can further comprise a protruded portion connected to the base portion. When the base portion is disposed (e.g., affixed or attached) along the edge of the recessed receptacle, the protruded portion is configured to cover, at least partially, a corner of the target location and align with a lateral surface of the sample plate to require the sample plate to be loaded at the target location in a correct orientation.

It should be appreciated that the geometry of the protruded portion of the alignment device may vary based on the geometry of the sample plate. It should also be appreciated that the alignment device can be secured to the scientific instrument via various means and can have other shapes. In some of the depicted examples, the protruded portion of the alignment device is secured to the scientific instrument by securing the base portion of the alignment device to the scientific instrument. The base portion can be secured by other means, such as using adhesive. The base portion can be of any other shape as long as it provides a mechanism for securing the alignment device to the scientific instrument. In some embodiments, the alignment device does not include any base portion. Instead, the protruded portion can be secured to the system at the right location via any appropriate means such that it aligns with the sample plate if the sample plate is correctly loaded and obstructs at least a portion of the sample plate if the sample plate is incorrectly loaded. It should also be appreciated that the location at the alignment device is installed may be changed, as described in detail below.

While the alignment mechanism may be a standalone device separate from the scientific instrument, it should be appreciated that the alignment mechanism can be an integral part of the scientific instrument or system. In some embodiments, the alignment mechanism is part of the target location, e.g., the receptacle, container, or surface area for accommodating a sample plate. For example, an exemplary receptacle of a scientific instrument can comprise a bottom configured to fit a bottom of the sample plate. The receptacle can further comprise a protruded portion covering at least partially a corner of an opening of the receptacle. When the sample plate is loaded in a correct orientation, the protruded portion is configured to align with a lateral cutoff of the sample plate to allow the sample plate to be placed onto the bottom of the receptacle. When the sample plate is loaded in an incorrect orientation, the protruded portion is configured to prevent the sample plate from being plated onto the bottom of the receptacle.

With the alignment mechanism, it is not possible to load the sample plate in an incorrect orientation. The alignment device requires the sample plate to be loaded only if it is in the correct orientation and may prevent the sample plate from being loaded when it is in an incorrect orientation. As a result, the alignment mechanism may eliminate the occurrence of loading errors, which are difficult to discover and expensive to remedy, and may improve the accuracy of the scientific instrument or system. The alignment mechanism also may eliminate the need for a second laboratory technician to check and confirm the correct loading of the sample plates into the scientific instrument, thus reducing the cost associated with operating the scientific instrument. Further, the attachment of the alignment device does not alter the functioning of the scientific instrument or system.

### Definitions

Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the terms “comprising” (and any form or variant of comprising, such as “comprise” and “comprises”), “having” (and any form or variant of having, such as “have” and “has”), “including” (and any form or variant of including, such as “includes” and “include”), or “containing” (and any form or variant of containing, such as “contains” and “contain”), are inclusive or open-ended and do not exclude additional, un-recited additives, components, integers, elements, or method steps.

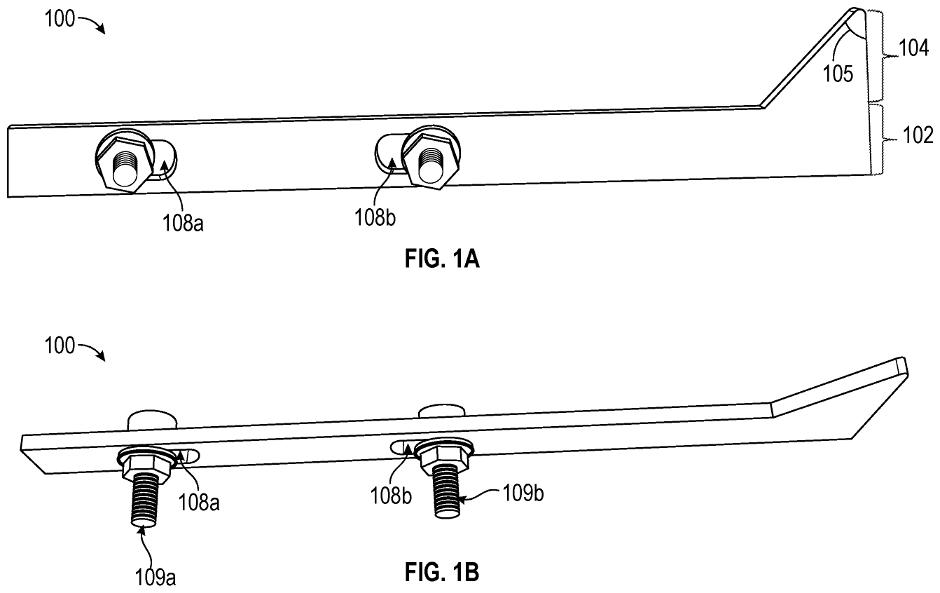

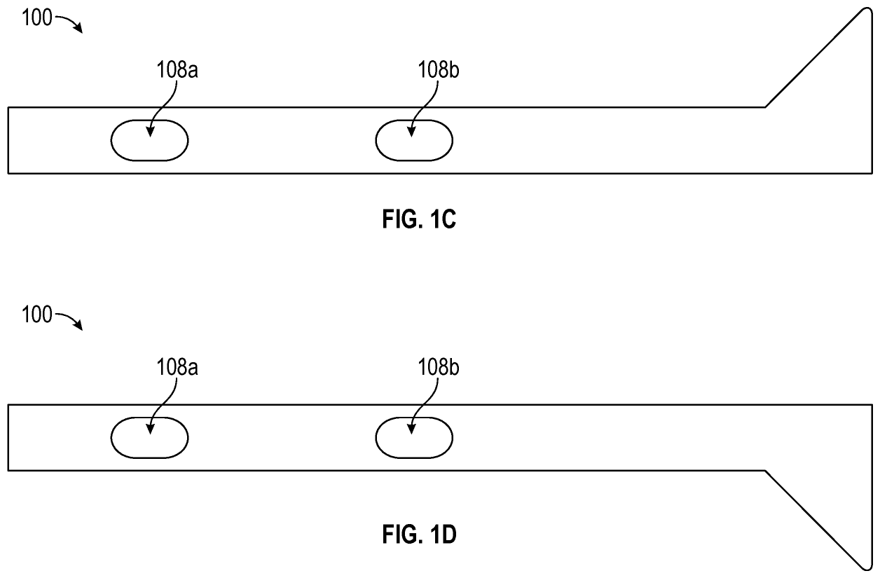

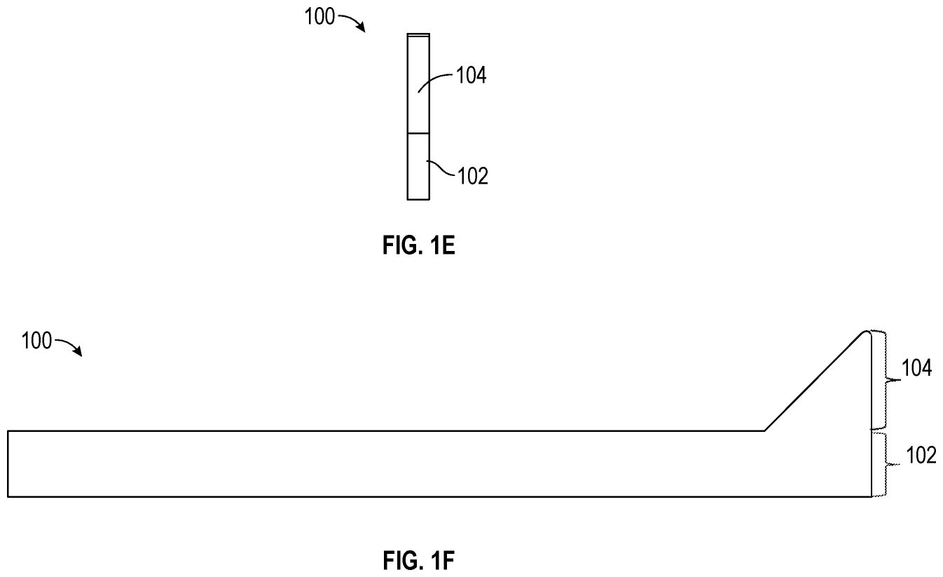

FIGS. 1A-1E illustrate an exemplary alignment device 100 for ensuring correct loading of a sample plate (not depicted), in accordance with some embodiments. With reference to FIG. 1A, the alignment device 100 comprises a base portion 102. In the depicted example, the base portion 102 comprises two apertures 108a and 108b for accommodating two fasteners 109a and 109b, such as bolt/nut sets, screws, rivets, welds, or any other means of attachment. The alignment device further comprises a protruded portion 104 connected to the base portion 102. In the depicted example, the protruded portion 104 is of a triangular shape having an angle 105 of 45°.

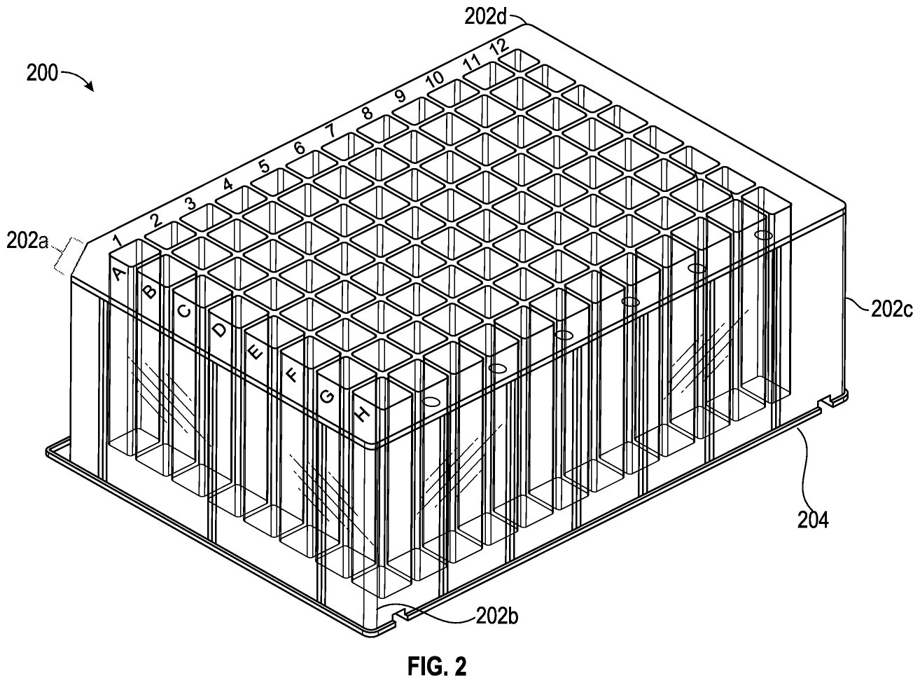

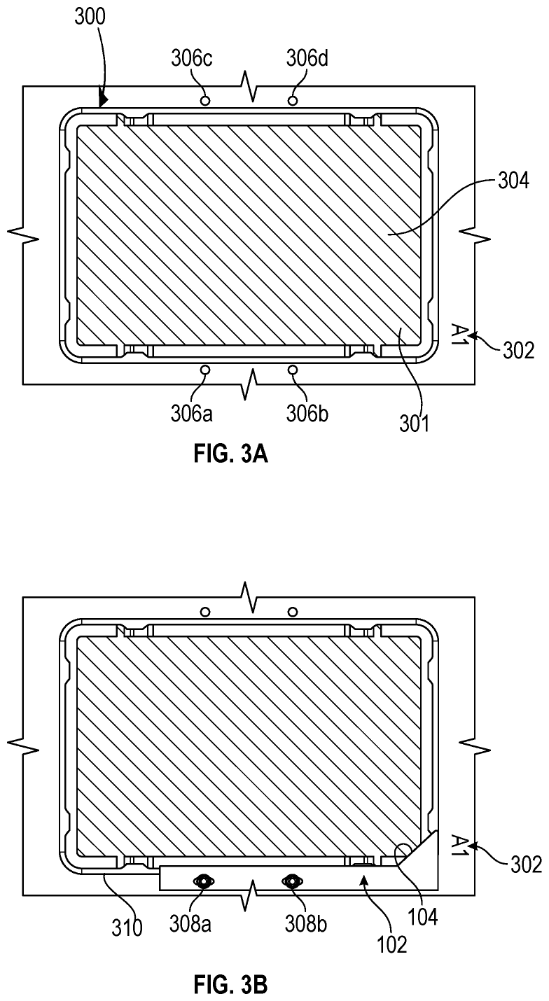

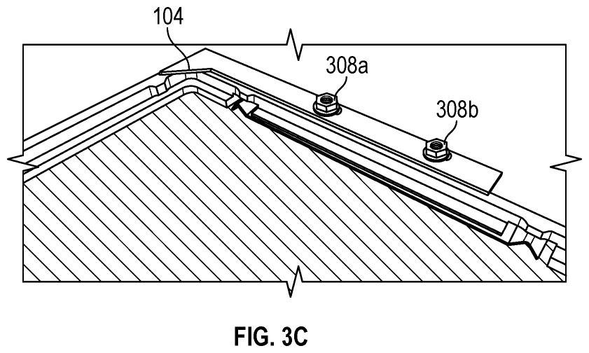

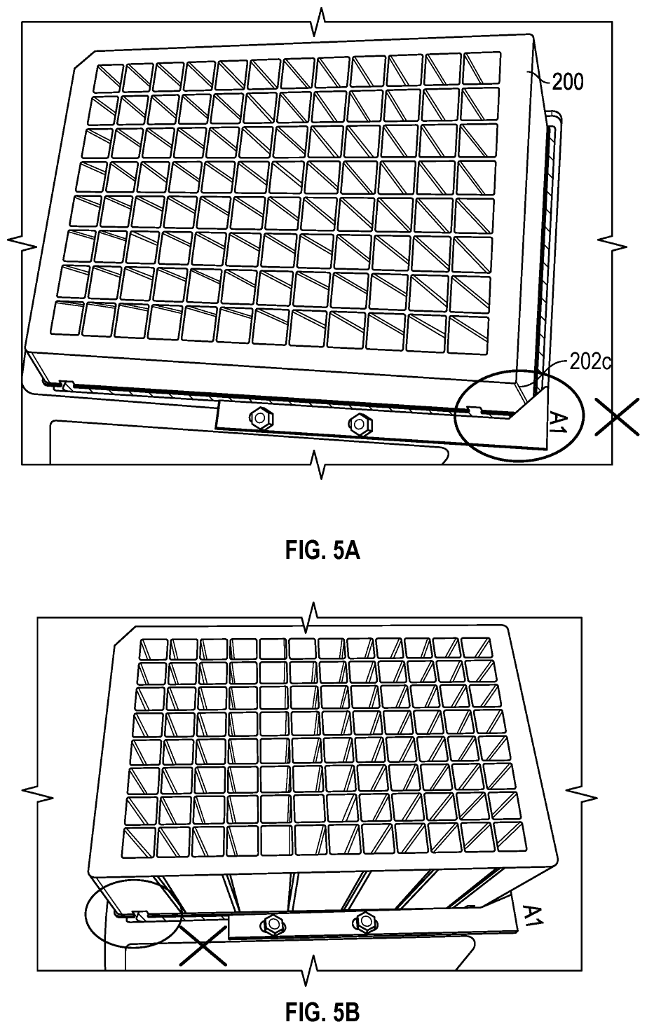

As described below, when the alignment device 100 is attached or affixed to a scientific instrument or system (e.g., a DNA extraction system) via the base portion 102, the protruded portion 104 is configured to cover at least partially a corner or an opening (e.g., corner 301 in FIG. 3A) of a target location (e.g., receptacle 300 in FIG. 3A) and align with a lateral surface of a cut-off (e.g., cut-off 202a in FIG. 2) of the sample plate to ensure the sample plate is being loaded into the receptacle in a correct orientation. It will be understood that the target location may be a receptacle (e.g., a recessed receptacle 300 in FIG. 3A), a container (e.g., a container that rests on a surface), a designated surface area, etc. The protruded portion 104 may cover at least partially a corner (e.g., corner 301 in FIG. 3A) of the target location to require the sample plate to be loaded at the target location in a correct orientation (e.g., FIGS. 5A-B). For case of understanding, the receptacle is one example of a target location for loading the sample plate on a scientific instrument. Further, it should be understood that, if the scientific instrument or system has multiple target locations, an alignment mechanism can be provided for each target location of the multiple target locations.

The alignment device 100 can be comprised or composed of any rigid material, including any rigid material that is resistant to corrosion and/or rust. In some embodiments, the alignment device is made of stainless steel. In some embodiments, the alignment device is made of plastic or resin. The alignment device can be manufactured using any suitable techniques. In some embodiments, the alignment device is manufactured via additive manufacturing techniques (e.g., 3D printing techniques).

In the depicted examples in FIGS. 1A-E, the protruded portion 104 of the alignment device is secured to the scientific instrument by securing the base portion 102 of the alignment device to the scientific instrument via apertures 108a-b and fasteners 109a-b. However, the base portion can be secured in other means, such as using adhesive. FIG. 1F provides depicts a top view of a variation of the exemplary alignment device 100 without apertures 108a-b. The device can be secured, for example, by affixing the base portion using adhesive.

FIG. 2 illustrates an exemplary sample plate 200, in accordance with some embodiments. As shown, the bottom 204 of the sample plate 200 is rectangular in shape. The sample plate 200 includes an array of slots for containing biological samples. In the depicted example, the rows of the array are labeled A, B, C, D, E, F, G, and H, while the columns of the array are labeled 1-12. The labels allow identification of a specific slot. For example, the first slot in the sample plate at the top left corner of the array can be identified as the A1 slot.

In the depicted example, the shape of the sample plate 200 is designed to provide a visual indicator as to the correct orientation of the sample plate. Specifically, with reference to FIG. 2, the corner at each of the lateral edges 202b, 202c, and 202d is 90°. But, the sample plate 200 also includes a lateral cutoff/surface 202a to indicate the location of the top left position of the array. Despite the cutoff/surface 202a serving as a visual indicator, however, it may be easily overlooked and nothing prevents the user from loading the sample plate 200 in a scientific instrument or system an incorrect orientation (e.g. incorrectly rotated by 180 degrees).

FIG. 3A illustrates an exemplary receptacle 300 of a scientific instrument or system that is configured to accommodate a sample plate, in accordance with some embodiments. The receptacle can be part of any scientific instrument or system, such as a DNA extractor system. In some embodiments, the receptacle is configured to accommodate a sample plate containing one or more biological samples (e.g., sample plate 200 in FIG. 2). After the sample plate is loaded into the receptacle, the content of the sample plate can be analyzed and/or processed by the system.

In the depicted example, the receptacle 300 is a recessed receptacle having a recessed bottom 304. A plurality of receptacle apertures, 306a, 306b, 306c, and 306d, are provided around the receptacle. A visual indicator 302 (“A1”) is provided at a corner of the receptacle to indicate the orientation in which the sample plate should be loaded. Specifically, when loading the plate, a user should make sure that the top left position of the array where the A1 slot is located is aligned with the visual indicator 302. It should be understood that the receptacle is only an example of a target location for accommodating a sample plate, and that a sample plate can be loaded at any type of target location, such as a container or a planar surface area. For example, the target location may be a planar surface of a scientific instrument and have the same size and shape as the recessed bottom 304.

However, as discussed above, despite the visual indicators (202a in FIGS. 2 and 302 in FIG. 3A), a user may overlook both visual indicators. Because the bottom of the sample plate 204 and the bottom of the receptacle 304 are both rectangular, nothing prevents the user from loading the sample plate into the receptacle in an incorrect orientation (e.g., off by 180 degrees) without the alignment mechanism described herein.

FIG. 3B illustrates the exemplary alignment device 100 installed on the scientific instrument or system to ensure correct loading of sample plates, in according with some embodiments. The base portion 102 is disposed (e.g., affixed or attached) along the edge 310 of the recessed receptacle via two fasteners 308a and 308b. In the depicted example, the receptacle apertures on the base portion 102 (e.g., 108a and 108b in FIG. 1A) are aligned with the receptacle apertures 306a and 306b (FIG. 3A) along the bottom edge of the receptacle, and two bolt/nut sets are used to secure the base portion 102 along the edge of the receptacle 300.

As shown in FIG. 3B, when the base portion 102 is disposed (e.g., affixed or attached) along the edge of the recessed receptacle 300, the protruded portion 104 covers a corner (corner 301 in FIG. 3A) of the recessed receptacle (i.e., the corner aligned with the visual indicator 302). FIG. 3C shows the installed alignment device from a different perspective, in accordance with some embodiments. The alignment device may be flush along the edge of the target location, but might not extend up to the edge (e.g., there may be a distance between the edge and an edge of the alignment device). As described below, the edge of the protruded portion 104 is configured to align with a lateral surface of the sample plate to ensure the sample plate will be loaded into the recessed receptacle in a correct orientation.

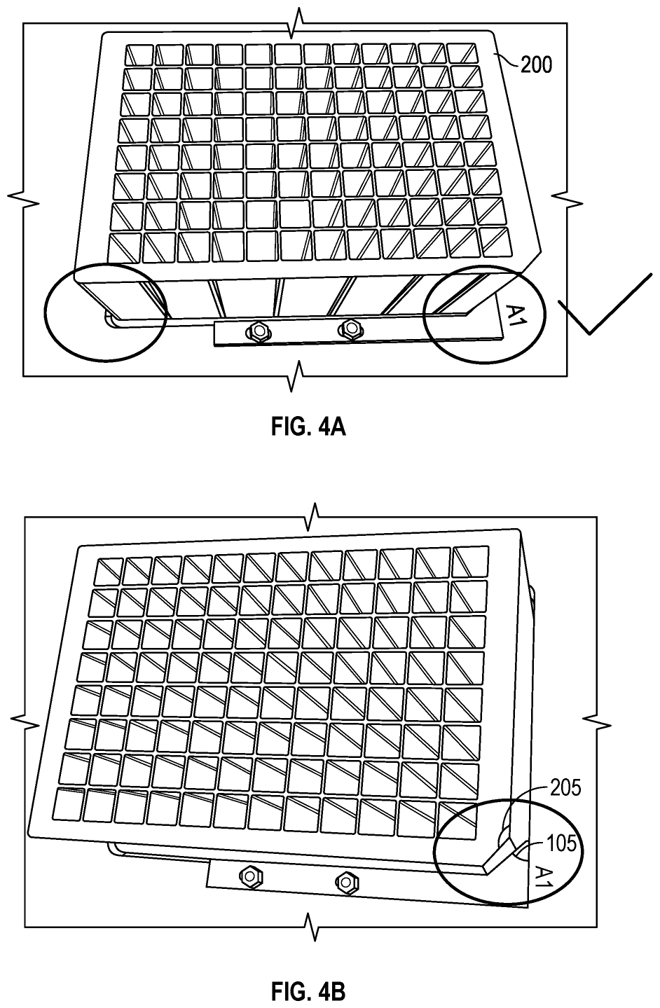

FIGS. 4A and 4B illustrate how the alignment device 100 permits the sample plate to fit into the receptacle when the sample plate 200 is in the correct orientation, in accordance with some embodiments. As shown, when the sample plate 200 is loaded in the correct orientation, the lateral cut-off of the sample plate 200 aligns with the edge of the protruded portion 104 of the alignment device 100. Specifically, as shown in FIG. 4B, the angle 105 of the protruded portion is supplementary to the angle 205 of the cut-off of the sample plate. Accordingly, the alignment device 100 does not block the sample plate 200 and the sample plate 200 can fit into the receptacle 300 as if the alignment device 100 is not present.

FIGS. 5A and 5B illustrate how the alignment device 100 ensures the sample plate 200 fits into the receptacle 300 only when the sample plate 200 is in a correct orientation. As shown, when the sample plate 200 is loaded in an incorrect orientation, the lateral edge of the sample plate 202c, which has a right-angle corner, does not align with the protruded portion 104 of the alignment device 100. As shown in FIG. 5B, due to the misalignment with the alignment device 100, the other corners of the sample plate 200 are unable to fit into the receptacle 300.

It should be appreciated that the geometry of the protruded portion 104 of the alignment device 100 may vary based on the geometry of the sample plate 300 and/or corners of the sample plate 300. In the depicted example in FIG. 4B, the angle 105) (45°) of the protruded portion 104 is supplementary to the angle 205) (135°) at the cut-off of the sample plate 300. But if the angle 205 is different from the angle shown in FIG. 4B, the angle 105 of the protruded portion would need to be different such that it is supplementary to the angle 205. For example, if the angle 205 is 120°, the angle 105 would need to be 60°. As another example, if the angle 205 is 145°, the angle 105 would need to be 35°. Further, the shape of the sample plate 300 may be different, and the protruded portion 104 of the alignment device 100 can be designed to have a complementary shape to the shape of the sample plate 300. For example, if the top left corner of the sample plate 300 is of a concave shape, the protruded portion 104 of the alignment device 100 needs to have a convex shape such that alignment can be achieved.

It should also be appreciated that the alignment device can be secured to the scientific instrument via other means and can have other shapes. In the depicted examples, the protruded portion of the alignment device is secured to the scientific instrument by securing the base portion of the alignment device to the scientific instrument. The base portion can be secured in other means, such as using adhesive. The base portion can be of any other shape as along as it provides a mechanism for securing the alignment device to the scientific instrument. In some embodiments, the alignment device does not include any base portion. Instead, the protrude portion itself can be secured to the system at the right location via any appropriate means such that it aligns the sample plate if it is correctly loaded and obstructs at least a portion of the sample plate if it is incorrectly loaded.

It should also be appreciated that the location at the alignment device is installed may be changed. For example, if a different corner of the sample plate has the lateral cut-off (e.g., at 202c), the alignment device would need to be installed at a different location around the receptacle such that the protruded portion aligns with the cut-off of the sample plate when the plate is correctly loaded.

It should also be appreciated that, if the sample plate comprises multiple lateral cutoffs, the alignment device can cover multiple corners of the target location.

While the alignment device may be a standalone device separate from the scientific instrument in the depicted examples, it should be appreciated that the alignment device can be an integral part of the scientific instrument. Specifically, a receptacle of the scientific instrument can comprise an element similar to the protruded portion of the alignment device to cover a corner or an opening of the receptacle to require the sample plate to be loaded in a correct orientation.

In the depicted example in FIGS. 3A and 3B, the receptacle apertures 308a-d may be originally designed for a different purpose than to secure the alignment device. For example, the screens apertures may be originally designed to accommodate screws to affix springs around the receptacle. Because the alignment device is fastened using the same receptacle apertures, longer screws may be needed to achieve both purposes of affixing the springs and affixing the alignment device.

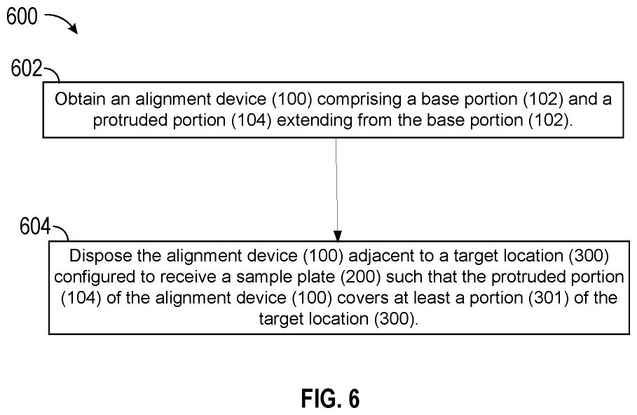

FIG. 6 illustrates an exemplary process 600 for installing an alignment device (e.g., alignment device 100) for ensuring correct loading of a sample plate (e.g. sample plate 200) into a target location (e.g., target location 300), in accordance with some embodiments. In the process 600, the alignment device may comprise a base portion (e.g., base portion 102) and a protruded portion (e.g., protruded portion 104). The protruded portion may be connected to and/or may extend from the base portion. In some embodiments, the protruded portion may have a triangular shape, an angle of which may be about 45°. The base portion and the protruded portion may be parts of a single component. In some embodiments of the process 600, the alignment device may comprise rigid, corrosion-resistant materials such as stainless steel, plastic, and/or resin.

The target location in process 600 may include a recessed receptacle disposed in a DNA extractor system, a container configured to receive the sample plate, and/or the planar surface configured to receive a sample plate. In some embodiments, the sample plate may be configured to contain one or more biological samples (e.g., tumor samples, tissue samples, blood samples, etc.).

The process 600 may be performed manually (e.g., by a technician) and/or automatically (e.g., by a robotic device). At block 602, the alignment device comprising the base portion and the protruded portion may be obtained. Obtaining the alignment device may comprise forming the alignment device via one or more additive manufacturing processes. At block 604, the alignment device may be disposed adjacent to the target location such that the protruded portion of the alignment device covers at least a portion (e.g., covered portion 301) of the target location.

Disposing the alignment device adjacent to the target location may comprise disposing the base portion of the alignment device along an edge of the target location. In some embodiments, the base portion of the alignment device may be affixed/attached along the edge of the target location. The base portion may be affixed/attached with one or more fasteners (e.g., fasteners 109a-b) and/or with an adhesive. In some embodiments, disposing the alignment device adjacent may comprise inserting one faster of the one or more fasteners into each of one or more apertures (e.g. apertures 108a-b) of the alignment device. After a fastener has been inserted into each of the one or more apertures, the alignment device may be moved relative to the fasteners in each of the apertures. Once the alignment device has been moved to a desired position relative to the target location, the position of the alignment device may be secured.

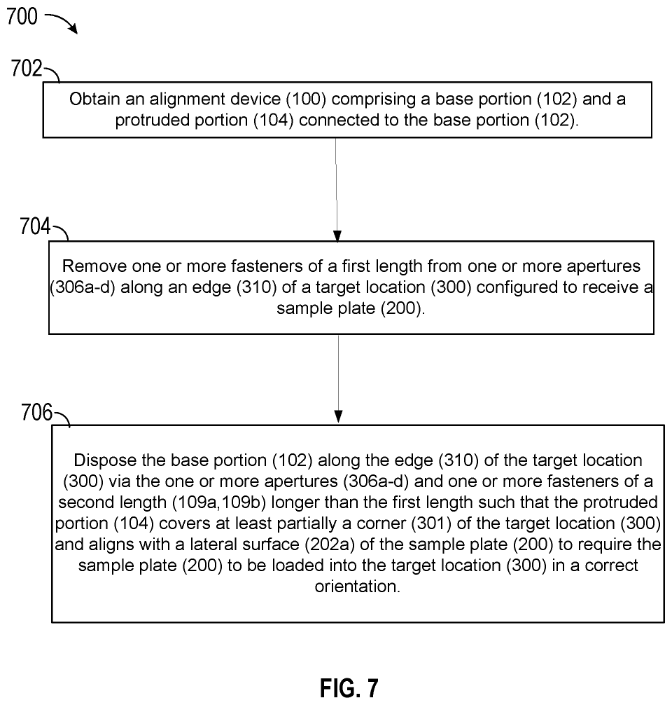

FIG. 7 illustrates an exemplary process 700 for installing the alignment device (e.g., alignment device 100) for ensuring correct loading of a sample plate (e.g., sample plate 200) into a target location (e.g., target location 200) which comprises one or more apertures in accordance with some embodiments. In the process 700, the alignment device may comprise a base portion (e.g., base portion 102) and a protruded portion (e.g., protruded portion 104). The protruded portion may be connected to and/or may extend from the base portion. In some embodiments, the protruded portion may have a triangular shape, an angle of which may be about 45°. The base portion and the protruded portion may be parts of a single component. In some embodiments of the process 700, the alignment device may comprise rigid, corrosion-resistant materials such as stainless steel, plastic, and/or resin.

The target location in the process 700 may include a recessed receptacle disposed in a DNA extractor system, a container configured to receive the sample plate, and/or the planar surface configured to receive a sample plate. In some embodiments, the sample plate may be configured to contain one or more biological samples (e.g., tumor samples, tissue samples, blood samples, etc.). In the process 700, the target location comprises one or more apertures (e.g., apertures 306a-d) along an edge (e.g., edge 310) of the target location. The apertures may hold one or more fasteners (e.g., screws) of a first length.

The process 700 may be performed manually (e.g., by a technician) and/or automatically (e.g., by a robotic device). At block 702, the alignment device comprising the base portion and the protruded portion connected to the base portion may be obtained. Obtaining the alignment device may comprise forming the alignment device via one or more additive manufacturing processes. At block 704, the one or more fasteners of the first length may be removed from the one or more apertures along the edge of the target location. At block 706, the alignment device may be disposed along the edge of the target location by disposing the base portion along the edge via the one or more apertures along the edge of the target location. The base portion may be disposed along the edge using one or more fasteners of a second length (e.g., fasteners 109a-b), wherein the second length is greater than the first length. In some embodiments, the one or more fasteners of the second length may be screws. This may cause the protruded portion of the alignment device to at least partially cover a corner (e.g., corner 301) of the target location and to align with a lateral surface (e.g., lateral surface 202a) of the sample plate. The alignment of the protruded portion with the lateral surface of the sample plate may ensure the sample plate is being loaded into the target location in a correct orientation.

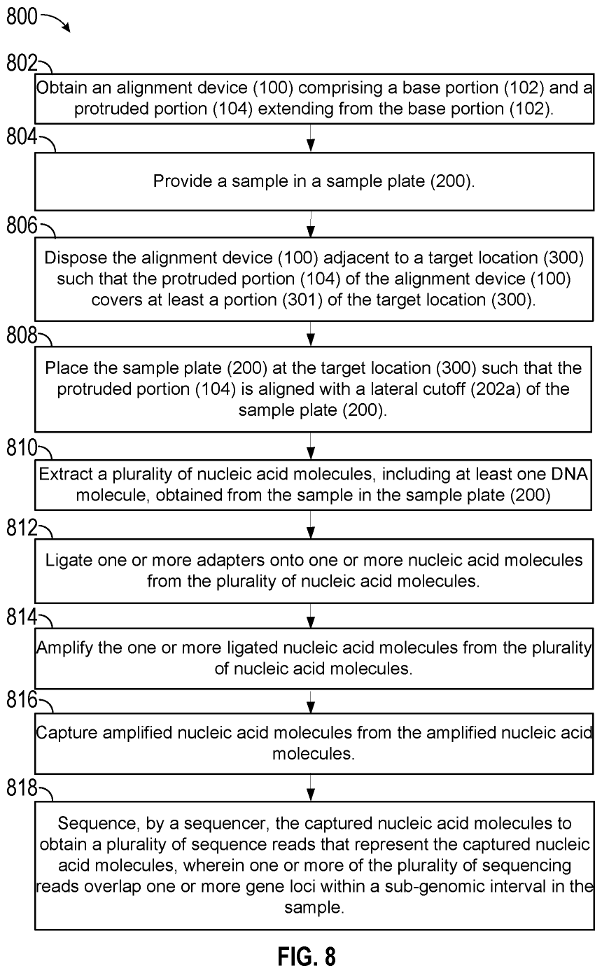

FIG. 8 illustrates an exemplary process 800 for installing an alignment device (e.g., alignment device 800) for ensuring correct loading of a sample plate (e.g., sample plate 200) in a target location (e.g., target location 300) and then sequencing a sample of nucleic acid molecules using the correctly loaded sample plate, in accordance with some embodiments. In the process 800, the alignment device may comprise a base portion (e.g., base portion 102) and a protruded portion (e.g., protruded portion 104). The protruded portion may be connected to and/or may extend from the base portion. In some embodiments, the protruded portion may have a triangular shape, an angle of which may be about 45°. The base portion and the protruded portion may be parts of a single component. In some embodiments of the process 800, the alignment device may comprise rigid, corrosion-resistant materials such as stainless steel, plastic, and/or resin.

The target location in the process 800 may include a recessed receptacle disposed in a DNA extractor system, a container configured to receive the sample plate, and/or the planar surface configured to receive a sample plate. In some embodiments, the sample plate may be configured to contain one or more biological samples (e.g., tumor samples, tissue samples, blood samples, etc.).

The process 800 may be performed manually (e.g., by a technician) and/or automatically (e.g., by a robotic device). At block 802, the alignment device comprising the base portion and the protruded portion connected to the base portion may be obtained. Obtaining the alignment device may comprise forming the alignment device via one or more additive manufacturing processes. At block 804, a sample may be provided in a sample plate (e.g., sample plate 200). In some embodiments the sample may be a biological sample. At block 806, the alignment device may be disposed adjacent to the target location such that the protruded portion of the alignment device covers at least a portion (e.g., covered portion 301) of the target location.

Disposing alignment device adjacent to the target location may comprise disposing the base portion of the alignment device along an edge of the target location. In some embodiments, the base portion may be affixed/attached along the edge of the target location. The base portion may be affixed with one or more fasteners (e.g., fasteners 109a-b) and/or with an adhesive. In some embodiments, disposing the alignment device adjacent may comprise inserting one faster of the one or more fasteners into each of one or more apertures (e.g. apertures 108a-b) of the alignment device. After a fastener has been inserted into each of the one or more apertures, the alignment device may be moved relative to the fasteners in each of the apertures. Once the alignment device has been moved to a desired position relative to the target location, the position of the alignment device may be secured.

At block 808, after the alignment device has been disposed adjacent to the target location, the sample plate may be placed at the target location such that a lateral cutoff (e.g., lateral cutoff 202a) of the sample plate aligns with the protruded portion of the alignment device. After the sample plate has been placed in the target location in the correct orientation, the sequencing process can be initiated. At block 810, a plurality of nucleic acid molecules may be obtained from the sample contained in the sample plate. In some embodiments, the plurality of nucleic acid molecules obtained from the sample may comprise at least one DNA molecule. At block 812, one or more adapters may be ligated onto one or more nucleic acid molecules from the plurality of nucleic acid molecules. At block 814, one or more of the ligated nucleic acid molecules from the plurality of nucleic acid molecules may be amplified. At block 816, amplified nucleic acid molecules may be captured. Finally, at block 818, the captured nucleic acid molecules may be sequenced by a sequencer in order to obtain a plurality of sequence reads that represent the captured nucleic acid molecules. In some embodiments of the process 800, one or more of the plurality of sequencing reads may overlap one or more gene loci within a sub-genomic interval in the sample.

The disclosed systems and methods may be used with any of a variety of samples. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor nucleic acid molecules and non-tumor nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor nucleic acid molecules may be derived from a circulating tumor DNA (ctDNA) fraction of the liquid biopsy sample while the non-tumor nucleic acid molecules may be derived from a non-tumor, cell-free DNA (cfDNA) fraction of the liquid biopsy sample.

In some instances, the disclosed methods and systems may be used to diagnose (or as part of a diagnosis of) the presence of disease or other condition (e.g., cancer, genetic disorders (such as Down Syndrome and Fragile X), neurological disorders, or any other disease type where detection of variants, e.g., copy number alternations, are relevant to diagnosing, treating, or predicting said disease) in a subject (e.g., a patient). In some instances, the disclosed methods may be applicable to diagnosis of any of a variety of cancers as described elsewhere herein.

In some instances, the disclosed methods and systems may be used to select an appropriate therapy or treatment (e.g., an anti-cancer therapy or anti-cancer treatment) for a subject. In some instances, for example, the anti-cancer therapy or treatment may comprise use of a poly (ADP-ribose) polymerase inhibitor (PARPi), a platinum compound, chemotherapy, radiation therapy, a targeted therapy (e.g., immunotherapy), surgery, or any combination thereof.

In some instances, the disclosed methods and systems may be used in treating a disease (e.g., a cancer) in a subject. For example, in response to analyzing the samples in the sample plate using any of the methods disclosed herein, an effective amount of an anti-cancer therapy or anti-cancer treatment may be administered to the subject.

In some instances, the methods and systems can further include administering or applying a treatment or therapy (e.g., an anti-cancer agent, anti-cancer treatment, or anti-cancer therapy) to the subject based on the generated genomic profile. An anti-cancer agent or anti-cancer treatment may refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

### Samples

The disclosed methods and systems may be used with any of a variety of samples (also referred to herein as specimens) comprising nucleic acids (e.g., DNA or RNA) that are collected from a subject (e.g., a patient). Examples of a sample include, but are not limited to, a tumor sample, a tissue sample, a biopsy sample (e.g., a tissue biopsy, a liquid biopsy, or both), a blood sample (e.g., a peripheral whole blood sample), a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces (or stool) sample, or other body fluid, secretion, and/or excretion sample (or cell sample derived therefrom). In certain instances, the sample may be frozen sample or a formalin-fixed paraffin-embedded (FFPE) sample.

In some instances, the sample may be collected by tissue resection (e.g., surgical resection), needle biopsy, bone marrow biopsy, bone marrow aspiration, skin biopsy, endoscopic biopsy, fine needle aspiration, oral swab, nasal swab, vaginal swab or a cytology smear, scrapings, washings or lavages (such as a ductal lavages or bronchoalveolar lavages), etc.

In some instances, the sample is a liquid biopsy sample, and may comprise, e.g., whole blood, blood plasma, blood serum, urine, stool, sputum, saliva, or cerebrospinal fluid. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the sample may comprise one or more premalignant or malignant cells. Premalignant, as used herein, refers to a cell or tissue that is not yet malignant but is poised to become malignant. In certain instances, the sample may be acquired from a solid tumor, a soft tissue tumor, or a metastatic lesion. In certain instances, the sample may be acquired from a hematologic malignancy or pre-malignancy. In other instances, the sample may comprise a tissue or cells from a surgical margin. In certain instances, the sample may comprise tumor-infiltrating lymphocytes. In some instances, the sample may comprise one or more non-malignant cells. In some instances, the sample may be, or is part of, a primary tumor or a metastasis (e.g., a metastasis biopsy sample). In some instances, the sample may be obtained from a site (e.g., a tumor site) with the highest percentage of tumor (e.g., tumor cells) as compared to adjacent sites (e.g., sites adjacent to the tumor). In some instances, the sample may be obtained from a site (e.g., a tumor site) with the largest tumor focus (e.g., the largest number of tumor cells as visualized under a microscope) as compared to adjacent sites (e.g., sites adjacent to the tumor).

In some instances, the disclosed methods may further comprise analyzing a primary control (e.g., a normal tissue sample). In some instances, the disclosed methods may further comprise determining if a primary control is available and, if so, isolating a control nucleic acid (e.g., DNA) from said primary control. In some instances, the sample may comprise any normal control (e.g., a normal adjacent tissue (NAT)) if no primary control is available. In some instances, the sample may be or may comprise histologically normal tissue. In some instances, the method includes evaluating a sample, e.g., a histologically normal sample (e.g., from a surgical tissue margin) using the methods described herein. In some instances, the disclosed methods may further comprise acquiring a sub-sample enriched for non-tumor cells, e.g., by macro-dissecting non-tumor tissue from said NAT in a sample not accompanied by a primary control. In some instances, the disclosed methods may further comprise determining that no primary control and no NAT is available, and marking said sample for analysis without a matched control.

In some instances, samples obtained from histologically normal tissues (e.g., otherwise histologically normal surgical tissue margins) may still comprise a genetic alteration such as a variant sequence as described herein. The methods may thus further comprise re-classifying a sample based on the presence of the detected genetic alteration. In some instances, multiple samples (e.g., from different subjects) are processed simultaneously.

The disclosed methods and systems may be applied to the analysis of nucleic acids extracted from any of variety of tissue samples (or disease states thereof), e.g., solid tissue samples, soft tissue samples, metastatic lesions, or liquid biopsy samples. Examples of tissues include, but are not limited to, connective tissue, muscle tissue, nervous tissue, epithelial tissue, and blood. Tissue samples may be collected from any of the organs within an animal or human body. Examples of human organs include, but are not limited to, the brain, heart, lungs, liver, kidneys, pancreas, spleen, thyroid, mammary glands, uterus, prostate, large intestine, small intestine, bladder, bone, skin, etc.

In some instances, the nucleic acids extracted from the sample may comprise deoxyribonucleic acid (DNA) molecules. Examples of DNA that may be suitable for analysis by the disclosed methods include, but are not limited to, genomic DNA or fragments thereof, mitochondrial DNA or fragments thereof, cell-free DNA (cfDNA), and circulating tumor DNA (ctDNA). Cell-free DNA (cfDNA) is comprised of fragments of DNA that are released from normal and/or cancerous cells during apoptosis and necrosis, and circulate in the blood stream and/or accumulate in other bodily fluids. Circulating tumor DNA (ctDNA) is comprised of fragments of DNA that are released from cancerous cells and tumors that circulate in the blood stream and/or accumulate in other bodily fluids.

In some instances, DNA is extracted from nucleated cells from the sample. In some instances, a sample may have a low nucleated cellularity, e.g., when the sample is comprised mainly of erythrocytes, lesional cells that contain excessive cytoplasm, or tissue with fibrosis. In some instances, a sample with low nucleated cellularity may require more, e.g., greater, tissue volume for DNA extraction.

In some instances, the nucleic acids extracted from the sample may comprise ribonucleic acid (RNA) molecules. Examples of RNA that may be suitable for analysis by the disclosed methods include, but are not limited to, total cellular RNA, total cellular RNA after depletion of certain abundant RNA sequences (e.g., ribosomal RNAs), cell-free RNA (cfRNA), messenger RNA (mRNA) or fragments thereof, the poly (A)-tailed mRNA fraction of the total RNA, ribosomal RNA (rRNA) or fragments thereof, transfer RNA (tRNA) or fragments thereof, and mitochondrial RNA or fragments thereof. In some instances, RNA may be extracted from the sample and converted to complementary DNA (cDNA) using, e.g., a reverse transcription reaction. In some instances, the cDNA is produced by random-primed cDNA synthesis methods. In other instances, the cDNA synthesis is initiated at the poly (A) tail of mature mRNAs by priming with oligo(dT)-containing oligonucleotides. Methods for depletion, poly(A) enrichment, and cDNA synthesis are well known to those of skill in the art.

In some instances, the sample may comprise a tumor content (e.g., comprising tumor cells or tumor cell nuclei), or a non-tumor content (e.g., immune cells, fibroblasts, and other non-tumor cells). In some instances, the tumor content of the sample may constitute a sample metric. In some instances, the sample may comprise a tumor content of at least 5-50%, 10-40%, 15-25%, or 20-30% tumor cell nuclei. In some instances, the sample may comprise a tumor content of at least 5%, at least 10%, at least 20%, at least 30%, at least 40%, or at least 50% tumor cell nuclei. In some instances, the percent tumor cell nuclei (e.g., sample fraction) is determined (e.g., calculated) by dividing the number of tumor cells in the sample by the total number of all cells within the sample that have nuclei. In some instances, for example when the sample is a liver sample comprising hepatocytes, a different tumor content calculation may be required due to the presence of hepatocytes having nuclei with twice, or more than twice, the DNA content of other, e.g., non-hepatocyte, somatic cell nuclei. In some instances, the sensitivity of detection of a genetic alteration, e.g., a variant sequence, or a determination of, e.g., microsatellite instability, may depend on the tumor content of the sample. For example, a sample having a lower tumor content can result in lower sensitivity of detection for a given size sample.

In some instances, as noted above, the sample comprises nucleic acid (e.g., DNA, RNA (or a cDNA derived from the RNA), or both), e.g., from a tumor or from normal tissue. In certain instances, the sample may further comprise a non-nucleic acid component, e.g., cells, protein, carbohydrate, or lipid, e.g., from the tumor or normal tissue.

### Subjects

In some instances, the sample is obtained (e.g., collected) from a subject (e.g., patient) with a condition or disease (e.g., a hyperproliferative disease or a non-cancer indication) or suspected of having the condition or disease. In some instances, the hyperproliferative disease is a cancer. In some instances, the cancer is a solid tumor or a metastatic form thereof. In some instances, the cancer is a hematological cancer, e.g. a leukemia or lymphoma.

In some instances, the subject has a cancer or is at risk of having a cancer. For example, in some instances, the subject has a genetic predisposition to a cancer (e.g., having a genetic mutation that increases his or her baseline risk for developing a cancer). In some instances, the subject has been exposed to an environmental perturbation (e.g., radiation or a chemical) that increases his or her risk for developing a cancer. In some instances, the subject is in need of being monitored for development of a cancer. In some instances, the subject is in need of being monitored for cancer progression or regression, e.g., after being treated with an anti-cancer therapy (or anti-cancer treatment). In some instances, the subject is in need of being monitored for relapse of cancer. In some instances, the subject is in need of being monitored for minimum residual disease (MRD). In some instances, the subject has been, or is being treated, for cancer. In some instances, the subject has not been treated with an anti-cancer therapy (or anti-cancer treatment).

In some instances, the subject (e.g., a patient) is being treated, or has been previously treated, with one or more targeted therapies. In some instances, e.g., for a patient who has been previously treated with a targeted therapy, a post-targeted therapy sample (e.g., specimen) is obtained (e.g., collected). In some instances, the post-targeted therapy sample is a sample obtained after the completion of the targeted therapy.

In some instances, the patient has not been previously treated with a targeted therapy. In some instances, e.g., for a patient who has not been previously treated with a targeted therapy, the sample comprises a resection, e.g., an original resection, or a resection following recurrence (e.g., following a disease recurrence post-therapy).

### Cancers

In some instances, the sample is acquired from a subject having a cancer. Exemplary cancers include, but are not limited to, B cell cancer (e.g., multiple myeloma), melanomas, breast cancer, lung cancer (such as non-small cell lung carcinoma or NSCLC), bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, adenocarcinomas, inflammatory myofibroblastic tumors, gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancers, essential thrombocythemia, agnogenic myeloid metaplasia, hypereosinophilic syndrome, systemic mastocytosis, familiar hypercosinophilia, chronic cosinophilic leukemia, neuroendocrine cancers, carcinoid tumors, and the like.

In some instances, the cancer is a hematologic malignancy (or premaligancy). As used herein, a hematologic malignancy refers to a tumor of the hematopoietic or lymphoid tissues, e.g., a tumor that affects blood, bone marrow, or lymph nodes. Exemplary hematologic malignancies include, but are not limited to, leukemia (e.g., acute lymphoblastic leukemia (ALL), acute myeloid leukemia (AML), chronic lymphocytic leukemia (CLL), chronic myelogenous leukemia (CML), hairy cell leukemia, acute monocytic leukemia (AMOL), chronic myelomonocytic leukemia (CMML), juvenile myelomonocytic leukemia (JMML), or large granular lymphocytic leukemia), lymphoma (e.g., AIDS-related lymphoma, cutaneous T-cell lymphoma, Hodgkin lymphoma (e.g., classical Hodgkin lymphoma or nodular lymphocyte-predominant Hodgkin lymphoma), mycosis fungoides, non-Hodgkin lymphoma (e.g., B-cell non-Hodgkin lymphoma (e.g., Burkitt lymphoma, small lymphocytic lymphoma (CLL/SLL), diffuse large B-cell lymphoma, follicular lymphoma, immunoblastic large cell lymphoma, precursor B-lymphoblastic lymphoma, or mantle cell lymphoma) or T-cell non-Hodgkin lymphoma (mycosis fungoides, anaplastic large cell lymphoma, or precursor T-lymphoblastic lymphoma)), primary central nervous system lymphoma, Sézary syndrome, Waldenström macroglobulinemia), chronic myeloproliferative neoplasm, Langerhans cell histiocytosis, multiple myeloma/plasma cell neoplasm, myelodysplastic syndrome, or myelodysplastic/myeloproliferative neoplasm.

### Nucleic Acid Extraction and Processing

DNA or RNA may be extracted from tissue samples, biopsy samples, blood samples, or other bodily fluid samples using any of a variety of techniques known to those of skill in the art (see, e.g., Example 1 of International Patent Application Publication No. WO 2012/092426; Tan, et al. (2009), “DNA, RNA, and Protein Extraction: The Past and The Present”, J. Biomed. Biotech. 2009:574398; the technical literature for the Maxwell® 16 LEV Blood DNA Kit (Promega Corporation, Madison, WI); and the Maxwell 16 Buccal Swab LEV DNA Purification Kit Technical Manual (Promega Literature #TM333, Jan. 1, 2011, Promega Corporation, Madison, WI)). Protocols for RNA isolation are disclosed in, e.g., the Maxwell® 16 Total RNA Purification Kit Technical Bulletin (Promega Literature #TB351, August 2009, Promega Corporation, Madison, WI).

A typical DNA extraction procedure, for example, comprises (i) collection of the fluid sample, cell sample, or tissue sample from which DNA is to be extracted, (ii) disruption of cell membranes (i.e., cell lysis), if necessary, to release DNA and other cytoplasmic components, (iii) treatment of the fluid sample or lysed sample with a concentrated salt solution to precipitate proteins, lipids, and RNA, followed by centrifugation to separate out the precipitated proteins, lipids, and RNA, and (iv) purification of DNA from the supernatant to remove detergents, proteins, salts, or other reagents used during the cell membrane lysis step.

Disruption of cell membranes may be performed using a variety of mechanical shear (e.g., by passing through a French press or fine needle) or ultrasonic disruption techniques. The cell lysis step often comprises the use of detergents and surfactants to solubilize lipids the cellular and nuclear membranes. In some instances, the lysis step may further comprise use of proteases to break down protein, and/or the use of an RNase for digestion of RNA in the sample.

Examples of suitable techniques for DNA purification include, but are not limited to, (i) precipitation in ice-cold ethanol or isopropanol, followed by centrifugation (precipitation of DNA may be enhanced by increasing ionic strength, e.g., by addition of sodium acetate), (ii) phenol-chloroform extraction, followed by centrifugation to separate the aqueous phase containing the nucleic acid from the organic phase containing denatured protein, and (iii) solid phase chromatography where the nucleic acids adsorb to the solid phase (e.g., silica or other) depending on the pH and salt concentration of the buffer.

In some instances, cellular and histone proteins bound to the DNA may be removed either by adding a protease or by having precipitated the proteins with sodium or ammonium acetate, or through extraction with a phenol-chloroform mixture prior to a DNA precipitation step.

In some instances, DNA may be extracted using any of a variety of suitable commercial DNA extraction and purification kits. Examples include, but are not limited to, the QIAamp (for isolation of genomic DNA from human samples) and DNAeasy (for isolation of genomic DNA from animal or plant samples) kits from Qiagen (Germantown, MD) or the Maxwell® and ReliaPrep™ series of kits from Promega (Madison, WI).

As noted above, in some instances the sample may comprise a formalin-fixed (also known as formaldehyde-fixed, or paraformaldehyde-fixed), paraffin-embedded (FFPE) tissue preparation. For example, the FFPE sample may be a tissue sample embedded in a matrix, e.g., an FFPE block. Methods to isolate nucleic acids (e.g., DNA) from formaldehyde- or paraformaldehyde-fixed, paraffin-embedded (FFPE) tissues are disclosed in, e.g., Cronin, et al., (2004) Am J Pathol. 164(1):35-42; Masuda, et al., (1999) Nucleic Acids Res. 27(22):4436-4443; Specht, et al., (2001) Am J Pathol. 158(2):419-429; the Ambion RecoverAll™ Total Nucleic Acid Isolation Protocol (Ambion, Cat. No. AM1975, September 2008); the Maxwell® 16 FFPE Plus LEV DNA Purification Kit Technical Manual (Promega Literature #TM349, February 2011); the E.Z.N.A.® FFPE DNA Kit Handbook (OMEGA bio-tek, Norcross, GA, product numbers D3399-00, D3399-01, and D3399-02, June 2009); and the QIAamp® DNA FFPE Tissue Handbook (Qiagen, Cat. No. 37625, October 2007). For example, the RecoverAll™ Total Nucleic Acid Isolation Kit uses xylene at elevated temperatures to solubilize paraffin-embedded samples and a glass-fiber filter to capture nucleic acids. The Maxwell® 16 FFPE Plus LEV DNA Purification Kit is used with the Maxwell® 16 Instrument for purification of genomic DNA from 1 to 10 μm sections of FFPE tissue. DNA is purified using silica-clad paramagnetic particles (PMPs), and eluted in low elution volume. The E.Z.N.A.® FFPE DNA Kit uses a spin column and buffer system for isolation of genomic DNA. QIAamp® DNA FFPE Tissue Kit uses QIAamp® DNA Micro technology for purification of genomic and mitochondrial DNA.

In some instances, the disclosed methods may further comprise determining or acquiring a yield value for the nucleic acid extracted from the sample and comparing the determined value to a reference value. For example, if the determined or acquired value is less than the reference value, the nucleic acids may be amplified prior to proceeding with library construction. In some instances, the disclosed methods may further comprise determining or acquiring a value for the size (or average size) of nucleic acid fragments in the sample, and comparing the determined or acquired value to a reference value, e.g., a size (or average size) of at least 100, 200, 300, 400, 500, 600, 700, 800, 900, or 1000 base pairs (bps). In some instances, one or more parameters described herein may be adjusted or selected in response to this determination.

After isolation, the nucleic acids are typically dissolved in a slightly alkaline buffer, e.g., Tris-EDTA (TE) buffer, or in ultra-pure water. In some instances, the isolated nucleic acids (e.g., genomic DNA) may be fragmented or sheared by using any of a variety of techniques known to those of skill in the art. For example, genomic DNA can be fragmented by physical shearing methods, enzymatic cleavage methods, chemical cleavage methods, and other methods known to those of skill in the art. Methods for DNA shearing are described in Example 4 in International Patent Application Publication No. WO 2012/092426. In some instances, alternatives to DNA shearing methods can be used to avoid a ligation step during library preparation.

### Sequencing Methods

The methods and systems disclosed herein can be used in combination with, or as part of, a method or system for sequencing nucleic acids (e.g., a next-generation sequencing system) to generate a plurality of sequence reads that overlap one or more gene loci within a subgenomic interval in the sample and thereby determine, e.g., gene allele sequences at a plurality of gene loci. “Next-generation sequencing” (or “NGS”) as used herein may also be referred to as “massively parallel sequencing”, and refers to any sequencing method that determines the nucleotide sequence of either individual nucleic acid molecules (e.g., as in single molecule sequencing) or clonally expanded proxies for individual nucleic acid molecules in a high throughput fashion (e.g., wherein greater than 10³, 10⁴, 10⁵ or more than 10⁵ molecules are sequenced simultaneously).

Next-generation sequencing methods are known in the art, and are described in, e.g., Metzker, M. (2010) Nature Biotechnology Reviews 11:31-46, which is incorporated herein by reference. Other examples of sequencing methods suitable for use when implementing the methods and systems disclosed herein are described in, e.g., International Patent Application Publication No. WO 2012/092426. In some instances, the sequencing may comprise, for example, whole genome sequencing (WGS), whole exome sequencing, targeted sequencing, or direct sequencing. In some instances, sequencing may be performed using, e.g., Sanger sequencing. In some instances, the sequencing may comprise a paired-end sequencing technique that allows both ends of a fragment to be sequenced and generates high-quality, alignable sequence data for detection of, e.g., genomic rearrangements, repetitive sequence elements, gene fusions, and novel transcripts.

The disclosed methods and systems may be implemented using sequencing platforms such as the Roche 454, Illumina Solexa, ABI-SOLID, ION Torrent, Complete Genomics, Pacific Bioscience, Helicos, and/or the Polonator platform. In some instances, sequencing may comprise Illumina MiSeq sequencing. In some instances, sequencing may comprise Illumina HiSeq sequencing. In some instances, sequencing may comprise Illumina NovaSeq sequencing. Optimized methods for sequencing a large number of target genomic loci in nucleic acids extracted from a sample are described in more detail in, e.g., International Patent Application Publication No. WO 2020/236941, the entire content of which is incorporated herein by reference.

In certain instances, the disclosed methods comprise one or more of the steps of: (a) acquiring a library comprising a plurality of normal and/or tumor nucleic acid molecules from a sample; (b) simultaneously or sequentially contacting the library with one, two, three, four, five, or more than five pluralities of target capture reagents under conditions that allow hybridization of the target capture reagents to the target nucleic acid molecules, thereby providing a selected set of captured normal and/or tumor nucleic acid molecules (i.e., a library catch); (c) separating the selected subset of the nucleic acid molecules (e.g., the library catch) from the hybridization mixture, e.g., by contacting the hybridization mixture with a binding entity that allows for separation of the target capture reagent/nucleic acid molecule hybrids from the hybridization mixture, (d) sequencing the library catch to acquiring a plurality of reads (e.g., sequence reads) that overlap one or more subject intervals (e.g., one or more target sequences) from said library catch that may comprise a mutation (or alteration), e.g., a variant sequence comprising a somatic mutation or germline mutation; (e) aligning said sequence reads using an alignment method as described elsewhere herein; and/or (f) assigning a nucleotide value for a nucleotide position in the subject interval (e.g., calling a mutation using, e.g., a Bayesian method or other method described herein) from one or more sequence reads of the plurality.

In some instances, acquiring sequence reads for one or more subject intervals may comprise sequencing at least 1, at least 5, at least 10, at least 20, at least 30, at least 40, at least 50, at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1,000, at least 1,250, at least 1,500, at least 1,750, at least 2,000, at least 2,250, at least 2,500, at least 2,750, at least 3,000, at least 3,500, at least 4,000, at least 4,500, or at least 5,000 loci, e.g., genomic loci, gene loci, microsatellite loci, etc. In some instances, acquiring a sequence read for one or more subject intervals may comprise sequencing a subject interval for any number of loci within the range described in this paragraph, e.g., for at least 2,850 gene loci.

In some instances, acquiring a sequence read for one or more subject intervals comprises sequencing a subject interval with a sequencing method that provides a sequence read length (or average sequence read length) of at least 20 bases, at least 30 bases, at least 40 bases, at least 50 bases, at least 60 bases, at least 70 bases, at least 80 bases, at least 90 bases, at least 100 bases, at least 120 bases, at least 140 bases, at least 160 bases, at least 180 bases, at least 200 bases, at least 220 bases, at least 240 bases, at least 260 bases, at least 280 bases, at least 300 bases, at least 320 bases, at least 340 bases, at least 360 bases, at least 380 bases, or at least 400 bases. In some instances, acquiring a sequence read for the one or more subject intervals may comprise sequencing a subject interval with a sequencing method that provides a sequence read length (or average sequence read length) of any number of bases within the range described in this paragraph, e.g., a sequence read length (or average sequence read length) of 56 bases.

In some instances, acquiring a sequence read for one or more subject intervals may comprise sequencing with at least 100× or more coverage (or depth) on average. In some instances, acquiring a sequence read for one or more subject intervals may comprise sequencing with at least 100×, at least 150×, at least 200×, at least 250×, at least 500×, at least 750×, at least 1,000×, at least 1,500×, at least 2,000×, at least 2,500×, at least 3,000×, at least 3,500×, at least 4,000×, at least 4,500×, at least 5,000×, at least 5,500×, or at least 6,000× or more coverage (or depth) on average. In some instances, acquiring a sequence read for one or more subject intervals may comprise sequencing with an average coverage (or depth) having any value within the range of values described in this paragraph, e.g., at least 160×.

In some instances, acquiring a read for the one or more subject intervals comprises sequencing with an average sequencing depth having any value ranging from at least 100× to at least 6,000× for greater than about 90%, 92%, 94%, 95%, 96%, 97%, 98%, or 99% of the gene loci sequenced. For example, in some instances acquiring a read for the subject interval comprises sequencing with an average sequencing depth of at least 125× for at least 99% of the gene loci sequenced. As another example, in some instances acquiring a read for the subject interval comprises sequencing with an average sequencing depth of at least 4,100× for at least 95% of the gene loci sequenced.

In some instances, the relative abundance of a nucleic acid species in the library can be estimated by counting the relative number of occurrences of their cognate sequences (e.g., the number of sequence reads for a given cognate sequence) in the data generated by the sequencing experiment.

In some instances, the disclosed methods and systems provide nucleotide sequences for a set of subject intervals (e.g., gene loci), as described herein. In certain instances, the sequences are provided without using a method that includes a matched normal control (e.g., a wild-type control) and/or a matched tumor control (e.g., primary versus metastatic).

In some instances, the level of sequencing depth as used herein (e.g., an X-fold level of sequencing depth) refers to the number of reads (e.g., unique reads) obtained after detection and removal of duplicate reads (e.g., PCR duplicate reads). In other instances, duplicate reads are evaluated, e.g., to support detection of copy number alteration (CNAs).

### Systems

In some instances, the disclosed systems may further comprise a sequencer, e.g., a next generation sequencer (also referred to as a massively parallel sequencer). Examples of next generation (or massively parallel) sequencing platforms include, but are not limited to, Roche/454's Genome Sequencer (GS) FLX system, Illumina/Solexa's Genome Analyzer (GA), Illumina's HiSeq® 2500, HiSeq® 3000, HiSeq® 4000 and NovaSeq® 6000 sequencing systems, Life/APG's Support Oligonucleotide Ligation Detection (SOLID) system, Polonator's G.007 system, Helicos BioSciences' HeliScope Gene Sequencing system, ThermoFisher Scientific's Ion Torrent Genexus system, or Pacific Biosciences' PacBio® RS system.

In some instances, the disclosed systems may be used for analyzing any of a variety of samples as described herein (e.g., a tissue sample, biopsy sample, hematological sample, or liquid biopsy sample derived from the subject).

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

What is claimed is:

1. An alignment device comprising:
a base portion (102) configured to be disposed along an edge (310) of a target location (300);
a protruded portion (104) connected to the base portion (102), wherein, when the base portion (102) is disposed along the edge (310) of the target location (300), the protruded portion (104) is configured to:
cover at least partially a corner (301) of the target location (300), and
align with a lateral surface (202a) of a sample plate (200) to require the sample plate (200) to be loaded at the target location (300) in a correct orientation.

2. The alignment device of claim 1, wherein the base portion (102) is attached along the edge (310) of the target location (300).

3. The alignment device of claim 1, wherein the protruded portion (104) is of a triangular shape.

4. The alignment device of claim 3, wherein an angle (105) of the triangular shape is 45°.

5. The alignment device of any of claims 1-4, wherein the base portion (102) is configured to be disposed along the edge (310) of the target location (300) via one or more fasteners (109a, 109b).

6. The alignment device of claim 5, wherein the one or more fasteners (109a, 109b) comprise one or more bolt/nut sets, screws, rivets, welds, or any combination thereof.

7. The alignment device of claim 6, wherein the base portion (102) comprises one or more apertures (108a, 108b) configured to receive the one or more fasteners (109a, 109b).

8. The alignment device of claim 7, wherein the one or more apertures (108a, 108b) are configured to allow for a location of the alignment device to be adjustable.

9. The alignment device of claim 1, wherein the base portion (102) is configured to be disposed along the edge (310) of the target location via adhesive.

10. The alignment device of any of claims 1-9, wherein the target location (300) includes a recessed receptacle disposed in a DNA extractor system.

11. The alignment device of any of claims 1-10, wherein the sample plate (200) is adapted to contain one or more biological samples.

12. The alignment device of claim 11, wherein the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

13. The alignment device of any of claims 1-12, wherein the alignment device comprises a rigid material that is resistant to corrosion and/or rust.

14. The alignment device of claim 13, wherein the alignment device comprises stainless steel.

15. The alignment device of claim 13, wherein the alignment device comprises plastic or resin.

16. The alignment device of any of claims 1-15, wherein the alignment device is formed via one or more additive manufacturing techniques.

17. The alignment device of any of claims 1-16, wherein the base portion (102) and the protruded portion (104) are parts of a single component.

18. A receptacle of a scientific instrument for accommodating a sample plate, comprising:
a bottom (304) configured to fit a bottom (204) of the sample plate (200);
a protruded portion (104) covering at least partially a corner (301) of an opening of the receptacle, wherein:
when the sample plate (200) is loaded in a correct orientation, the protruded portion (104) is configured to align with a lateral cutoff (202a) of the sample plate (200) to allow the sample plate to be placed on the bottom (304) of the receptacle; and
when the sample plate (200) is loaded in an incorrect orientation, the protruded portion is configured to require the sample plate (200) to be loaded on the bottom (304) in a correct orientation.

19. The receptacle of claim 18, wherein the protruded portion (104) is part of an alignment device comprising a base portion (102) configured to be disposed along an edge (310) of the receptacle.

20. The receptacle of claim 19, wherein the base portion (102) is configured to be attached along the edge (310) of the receptacle.

21. The receptacle of claim 19, wherein the base portion (102) is configured to be disposed along the edge (310) of the receptacle via one or more fasteners (109a, 109b).

22. The receptacle of claim 21, wherein the one or more fasteners (109a, 109b) comprise one or more bolt/nut sets, screws, rivets, welds, or any combination thereof.

23. The receptacle of claim 22, wherein the base portion (102) comprises one or more apertures (108a, 108b) configured to receive one or more fasteners (109a, 109b).

24. The receptacle of claim 23, wherein the one or more apertures (108a, 108b) are configured to allow for a location of the alignment device to be adjustable.

25. The receptacle of any of claim 19, wherein the base portion (102) is configured to be disposed along the edge (310) of the receptacle via adhesive.

26. The receptacle of any of claims 19-25, wherein the alignment device comprises a rigid material that is resistant to corrosion.

27. The receptacle of claim 26, wherein the alignment device comprises stainless steel.

28. The receptacle of claim 27, wherein the alignment device comprises plastic or resin.

29. The receptacle of any of claims 19-28, wherein the alignment device is formed via one or more additive manufacturing techniques.

30. The receptacle of any of claims 19-29, wherein the base portion (102) and the protruded portion (104) are parts of a single component.

31. The receptacle of claim 18, wherein the protruded portion (104) is an integral part of the receptacle.

32. The receptacle of any of claims 18-31, wherein the bottom (304) of the receptacle is rectangular.

33. The receptacle of any of claims 18-32, wherein the protruded portion (104) is of a triangular shape.

34. The receptacle of claim 33, wherein an angle (105) of the triangular shape is 45°.

35. The receptacle of any of claims 18-34, wherein the receptacle is configured to be disposed in a DNA extractor system.

36. The receptacle of any of claims 18-35, wherein the sample plate (200) is adapted to contain one or more biological samples.

37. The receptacle of claim 36, wherein the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

38. A method for installing an alignment device (100) for enabling a sample plate (200) to be loaded at a target location (300) in a correct orientation, wherein the alignment device (100) comprises:
a base portion (102), and
a protruded portion (104) extending from the base portion (102), the method comprising:
disposing the alignment device (100) adjacent to the target location (300) such that the protruded portion (104) of the alignment device (100) covers at least a portion (301) of the target location (300).

39. The method of claim 38, wherein the protruded portion (104) is of a triangular shape.

40. The method of claim 39, wherein an angle (105) of the triangular shape is 45°.

41. The method of any of claims 38-40, wherein disposing the alignment device (100) comprises disposing the base portion (102) along an edge (310) of the target location (300).

42. The method of claim 41, wherein the base portion (102) is disposed along the edge (310) via adhesive.

43. The method of claim 41, wherein the base portion (102) is disposed along the edge (310) via one or more fasteners (109a, 109b).

44. The method of claim 43, wherein the one or more fasteners (109a, 109b) comprise one or more bolt/nut sets, screws, rivets, welds, or any combination thereof.

45. The method of claim 44, wherein the base portion comprises one or more apertures (108a, 108b) configured to receive the one or more fasteners (109a, 109b).

46. The method of claim 45, wherein the one or more apertures (108a, 108b) are configured to allow for a location of the alignment device (100) to be adjustable.

47. The method of claim 46, wherein disposing the alignment device (100) comprises:
inserting one fastener from the one or more fasteners (109a, 109b) into each of the one or more apertures (108a, 108b);
moving the alignment device (100) relative to the one or more fasteners (109a, 109b) in each of the one or more apertures (108a, 108b); and
securing, in response to the alignment device (100) being at a desired position relative to the target location (300), the position of the alignment device (100).

48. The method of claim 41, wherein the base portion (102) is configured to attach along the edge (310) of the target location (300).

49. The method of any of claims 38-48, wherein the target location (300) includes a recessed receptacle disposed in a DNA extractor system.

50. The method of any of claims 38-49, wherein the sample plate (200) is adapted to contain one or more biological samples.

51. The method of any of claims 50, wherein the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

52. The method of any of claims 38-51, wherein the alignment device (100) comprises a rigid material that is resistant to corrosion and/or rust.

53. The method of any of claims 52, wherein the alignment device (100) comprises stainless steel.

54. The method of any of claims 52, wherein the alignment device (100) comprises plastic or resin.

55. The method of any of claims 38-54, wherein the alignment device (100) is formed via one or more additive manufacturing techniques.

56. The method of any of claims 38-54, wherein the base portion (102) and the protruded portion (104) are parts of a single component.

57. A DNA extractor comprising:
a target location (300); and
an alignment device (100) comprising:
a base portion (102) configured to be disposed along an edge (310) of the target location (300);
a protruded portion (104) connected to the base portion (102), wherein, when the base portion (102) is disposed along the edge (310) of the target location (300), the protruded portion (104) is configured to:
cover at least partially a corner (301) of the target location (300), and
align with a lateral surface (202a) of a sample plate (200) to require the sample plate (200) to be loaded at the target location (300) in a correct orientation.

58. The DNA extractor of claim 57, wherein the protruded portion (104) is of a triangular shape.

59. The DNA extractor of claim 58, wherein an angle (105) of the triangular shape is 45°.

60. The DNA extractor of any of claims 56-59, wherein the base portion (102) is configured to be disposed along the edge (310) of the target location (300) via one or more fasteners (109a, 109b).

61. The DNA extractor of claim 60, wherein the one or more fasteners (109a, 109b) comprise one or more bolt/nut sets, screws, rivets, welds, or any combination thereof.

62. The DNA extractor of claim 61, wherein the base portion (102) comprises one or more apertures (108a, 108b) configured to receive the one or more fasteners (109a, 109b).

63. The DNA extractor of claim 62, wherein the one or more apertures (108a, 108b) are configured to allow for a location of the alignment device (100) to be adjustable.

64. The DNA extractor claim 57, wherein the base portion (102) is configured to be disposed along the edge (310) of the target location (300) via adhesive.

65. The DNA extractor of claim 57, wherein the base portion (102) is configured to attach along the edge (310) of the target location (300).

66. The DNA extractor of any of claims 57-65, wherein the target location (300) includes a recessed receptacle disposed in a DNA extractor system.

67. The DNA extractor of any of claims 57-66, wherein the target location (300) includes a container configured to receive the sample plate (200).

68. The DNA extractor of any of claims 57-67, wherein the target location (300) includes a planar surface configured to receive the sample plate (200).

69. The DNA extractor of any of claims 57-68, wherein the sample plate (200) is adapted to contain one or more biological samples.

70. The DNA extractor of claim 69, wherein the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

71. The DNA extractor of any of claims 57-70, wherein the alignment device (100) comprises a rigid material that is resistant to corrosion and/or rust.

72. The DNA extractor of claim 71, wherein the alignment device (100) comprises stainless steel.

73. The DNA extractor of claim 71, wherein the alignment device (100) comprises plastic or resin.

74. The DNA extractor of any of claims 57-73, wherein the alignment device (100) is formed via one or more additive manufacturing techniques.

75. The DNA extractor of any of claims 57-74, wherein the base portion (102) and the protruded portion (104) are parts of a single component.

76. A method for installing an alignment device (100) for requiring correct loading of a sample plate (200) into a target location (300),
wherein the target location (300) comprises one or more apertures (306a-d) along an edge (310) of the target location (300),
wherein the alignment device (100) comprises:
a base portion (102), and
a protruded portion (104) connected to the base portion (102),
the method comprising:
removing one or more fasteners of a first length from the one or more apertures;
disposing the base portion (102) along the edge (310) of the target location (300) via the one or more apertures (306a-d) and one or more fasteners of a second length (109a, 109b) longer than the first length such that the protruded portion (104) covers at least partially a corner (301) of the target location (300) and aligns with a lateral surface (202a) of the sample plate (200) to require the sample plate (200) to be loaded into the target location (300) in a correct orientation.

77. The method of claim 76, wherein the one or more fasteners of the first length are screws of the first length.

78. The method of claim 76 or 77, wherein the one or more fasteners of the second length are screws of the second length.

79. The method of any of claims 76-78, wherein the protruded portion (104) is of a triangular shape.

80. The method of claim 79, wherein an angle (105) of the triangular shape is 45°.

81. The method of any of claims 76-80, wherein the base portion (102) comprises one or more apertures (108a, 108b) configured to receive the one or more fasteners of the second length (109a, 109b).

82. The method of claim 81, wherein the one or more apertures (108a, 108b) are configured to allow for a location of the alignment device (100) to be adjustable.

83. The method of any of claims 76-82, wherein the target location (300) includes a recessed receptacle disposed in a DNA extractor system.

84. The method of any of claims 76-83, wherein the target location (300) includes a container configured to receive the sample plate (200).

85. The method of any of claims 76-84, wherein the target location (300) includes a planar surface configured to receive the sample plate (200).

86. The method of any of claims 76-85, wherein the sample plate (200) is adapted to contain one or more biological samples.

87. The method of claim 86, wherein the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

88. The method of any of claims 76-87, wherein the alignment device (100) comprises a rigid material that is resistant to corrosion and/or rust.

89. The method of claim 88, wherein the alignment device (100) comprises stainless steel.

90. The method of claim 88, wherein the alignment device (100) comprises plastic or resin.

91. The method of any of claims 76-90, wherein the alignment device (100) is formed via one or more additive manufacturing techniques.

92. The method of any of claims 76-91, wherein the base portion (102) and the protruded portion (104) are parts of a single component.

93. A method for extracting deoxyribonucleic acid (DNA) molecules from a sample from a subject using a DNA extraction device, the method comprising:
providing the sample in a sample plate (200);
disposing an alignment device (100) comprising a base portion (102) and a protruded portion (104) extending from the base portion (102) adjacent to a target location (300) on the DNA extraction device such that the protruded portion (104) of the alignment device (100) covers at least a portion (301) of the target location (300);
placing the sample plate (200) at the target location (300) such that the protruded portion (104) is aligned with a lateral cutoff (202a) of the sample plate (200);
extracting a plurality of nucleic acid molecules, including at least one DNA molecule, from the sample in the sample plate (200);
ligating one or more adapters onto one or more nucleic acid molecules from the plurality of nucleic acid molecules;
amplifying the one or more ligated nucleic acid molecules from the plurality of nucleic acid molecules;
capturing amplified nucleic acid molecules from the amplified nucleic acid molecules; and
sequencing, by a sequencer, the captured nucleic acid molecules to obtain a plurality of sequence reads that represent the captured nucleic acid molecules, wherein one or more of the plurality of sequencing reads overlap one or more gene loci within a subgenomic interval in the sample.

94. The method of claim 93, wherein the protruded portion (104) is of a triangular shape.

95. The method of claim 94, wherein an angle (105) of the triangular shape is 45°.

96. The method of any of claims 93-95, wherein disposing the alignment device (100) comprises disposing the base portion (102) along an edge (310) of the target location (300).

97. The method of claim 96, wherein the base portion (102) is disposed along the edge (310) via adhesive.

98. The method of claim 97, wherein the base portion (102) is disposed along the edge (310) via one or more fasteners (109a, 109b).

99. The method of claim 98, wherein the one or more fasteners (109a, 109b) comprise one or more bolt/nut sets, screws, rivets, welds, or any combination thereof.

100. The method of claim 99, wherein the base portion (102) comprises one or more apertures (108a, 108b) configured to receive the one or more fasteners.

101. The method of claim 100, wherein the one or more apertures (108a, 108b) are configured to allow for a location of the alignment device (100) to be adjustable.

102. The method of claim 101, wherein disposing the alignment device (100) comprises:
inserting one fastener from the one or more fasteners (109a, 109b) into each of the one or more apertures (108a, 108b);
moving the alignment device (100) relative to the one or more fasteners (109a, 109b) in each of the one or more apertures (108a, 108b); and
securing, in response to the alignment device (100) being at a desired position relative to the target location (300), the position of the alignment device (100).

103. The method of claim 96, wherein disposing the alignment device (100) comprises attaching the base portion (102) along the edge (310) of the target location.

104. The method of any of claims 93-103, wherein the target location (300) includes a recessed receptacle disposed in a DNA extractor system.

105. The method of any of claims 93-104, wherein the sample plate (200) is adapted to contain one or more biological samples.

106. The method of claim 105, wherein the one or more biological samples comprise a tumor sample, a tissue sample, a biopsy sample, a blood sample, a blood plasma sample, a blood serum sample, a lymph sample, a saliva sample, a sputum sample, a urine sample, a gynecological fluid sample, a circulating tumor cell (CTC) sample, a cerebral spinal fluid (CSF) sample, a pericardial fluid sample, a pleural fluid sample, an ascites (peritoneal fluid) sample, a feces or stool sample, or other body fluid, secretion, and/or excretion sample.

107. The method of any of claims 93-106, wherein the alignment device (100) comprises a rigid material that is resistant to corrosion and/or rust.

108. The method of claim 107, wherein the alignment device (100) comprises stainless steel.

109. The method of claim 107, wherein the alignment device (100) comprises plastic or resin.

110. The method of any of claims 93-109, wherein the alignment device (100) is formed via one or more additive manufacturing techniques.

111. The method of any of claims 93-109, wherein the base portion (102) and the protruded portion (104) are parts of a single component.
